(19) United States
(12) Patent Application Publication
(10) Pub. No.: US 2022/0401341 A1
(43) Pub. Date: Dec. 22, 2022

(54) Colored particles

(71) Applicant: SYMRISE AG, Holzminden (DE)

(72) Inventors: Jens-Michael Hilmer, Holzminden (DE); Sergej Elenschleger, Holzminden (DE); Rita Lages, Bodenwerder (DE); Ingo Reiss, Holzminden (DE)

(21) Appl. No.: 17/774,777

(22) PCT Filed: Nov. 6, 2019
(86) PCT No.: PCT/EP2019/080448

§ 371 Date: May 5, 2022

(51) Int. Cl. A61K 8/73; A61K 8/02

(52) U.S. Cl. CPC A61K 8/732; A61K 8/0283; A61K 2800/42; A61K 2800/10

(57) ABSTRACT

The present invention relates to color particles from an extrudate, the production thereof, color particles produced by the process and the use of the color particles for the production of foodstuffs, beverages, cosmetics, in particular oral hygiene products, pharmaceuticals and consumer articles, in particular washing and cleaning agents in liquid, gel-like or powdery form, animal food or animal care products and foods, beverages, cosmetics, in particular oral hygiene products, pharmaceuticals, consumer goods, in particular detergents and cleaning agents in liquid, gel-like or powdery form, animal food or animal care products comprising the color particles.

### FIELD OF INVENTION

The present invention relates to color particles from an extrudate, the production thereof, color particles produced by the process and the use of the color particles for the production of foodstuffs, beverages, cosmetics, in particular oral hygiene products, pharmaceuticals and consumer articles, in particular washing and cleaning agents in liquid, gel-like or powdery form, animal food or animal care products and foodstuffs, beverages, cosmetics, in particular oral hygiene products, pharmaceuticals and consumer goods, in particular washing and cleaning agents in liquid, gel-like or powdery form, animal food or animal care products, which comprise the color particles.

### STATE OF THE ART

In the cosmetics industry, swelling or partially soluble microparticles of synthetic plastics are widely added to cosmetic products for optical purposes (appearance or gloss) or to give consistency.

Aesthetic effects in particular play an important role in consumer acceptance of these compositions. As a rule, decorative effects are used to distinguish certain products on the market or to identify products with different properties.

In the field of dentifrices, which can be used for both therapeutic and cosmetic purposes, clear dentifrice products such as toothpastes and gels are known, for example, in which contrasting colored particles or flakes are incorporated. These particles provide an aesthetic effect that is perceived as pleasant by the consumer and encourages the use of the dentifrice, including by children.

Due to adverse health effects and environmental damage attributed to the above-described microparticles, also known as microbeads, more and more manufacturers of cosmetics and consumer products are abandoning the use of such plastic-based microparticles.

As an alternative to the above microbeads, natural-based particles are used in the manufacture of food, beverages and cosmetic products for the above purposes. For example, particles based on a natural carbohydrate matrix are known from the prior art.

U.S. Pat. No. 4,663,152 A discloses agglomerated speckles for incorporation into dentifrices comprising agglomerates of a water-insoluble powdered functional material and water-insoluble ethanol-soluble ethyl cellulose. Such sprinkles satisfactorily retain their integrity and identity during processing of the dentifrice but soften during storage after packaging of the dentifrice to allow the user to substantially brush his teeth without sensitivity. Despite this softening, the speckles retain their identity as speckles in the dentifrice. The speckles are particularly useful in translucent or transparent gel dentifrices.

EP 1 030 734 A1 describes an emulsion or dispersion of active material, for example aroma, encapsulated in an alginate matrix and its use in toothpastes.

U.S. Pat. No. 6,235,274 A discloses microparticles consisting of: (a) an olfactory active component (e.g., aroma or perfume component); (b) silica; and (c) a saccharide composition which is a mixture of mannitol and maltose. The microparticles are useful for enhancing, improving and/or imparting aroma and/or flavor over time in a controllably releasable manner for perfume compositions, perfumed articles, foods, chewing gums, beverages and the like. In particular, the use in antiperspirant/deodorant compositions is emphasized. There is no reference to the visibility of the particles in the product.

EP 1 361 803 A1 relates to a granular delivery system for flavoring or perfume compositions based on a matrix comprising at least one carbohydrate material with 1 to 7% prehydrated agar-agar. The disclosed system is particularly stable in aqueous environments and is capable of controlled release of a flavoring or odiferous substance encapsulated in the system.

EP 1 753 307 A1 discloses particulate compositions comprising controlled release particles, wherein flavor-containing fat having a melting point of at least 35° C. is dispersed in a gelatin matrix. The flavor-containing fat is released under specific conditions, such as under the influence of shear forces, heat and/or moisture. The release rate can be influenced by varying the relative amounts of gelatin and fat and the gelling strength of the gelatin. The composition described is suitable for imparting a long-lasting flavor impression.

EP 2 154 985 A1 describes an oily active agent delivery system comprising an extrudate of a melt emulsion, wherein the continuous phase of the emulsion comprises a matrix material and the dispersed phase comprises the oily active agent and an effective amount of a viscosity modifying ingredient, for example ethyl cellulose.

WO 2010/019587 A2 relates to oral care compositions comprising capsules containing flavorings and/or one or more active ingredients. The matrix of these capsules consists of chitosan, algin, agar or mixtures thereof.

WO 2010/115037 A2 has as its object non-aqueous dentifrice compositions with improved mouthfeel, foam and product stability. The non-aqueous compositions include combinations of carrageenan and/or carboxymethyl cellulose gums, glycerin, ethylene oxide/propylene oxide copolymers, and a bioactive glass such as calcium sodium phosphosilicate.

WO 2010/114549 A1 discloses a dentifrice composition comprising a plurality of granules and an orally acceptable carrier. The granules include at least one abrasive agent and at least one polymeric binder.

WO 2010/131207 A1 describes a process for preparing a granular release system comprising the following steps: (i) generating a melt emulsion consisting of a continuous phase and a dispersed active ingredient, wherein the continuous phase comprises trehalose and a carbohydrate, (ii) forcing the melt emulsion through a nozzle or orifice to form an extrudate, (iii) cooling and granulating the extrudate, and (iv) optionally drying the granules.

WO 2013/178638 A1 relates to encapsulated flavor particles comprising a particulate ingredient of vegetable raw materials, such as rice, tapioca, wheat, corn, sorghum, sago grains or peas, and a flavor coating comprising a sprayed emulsion of flavor and thermoreversible potato starch, wherein the thermoreversible potato starch comprises at least 80% by weight of amylopectin modified with amylomaltase enzyme. The coated particles provide good flavor release properties in consumer products such as chewing gums, beverages, foods, toothpastes, mouthwashes and the like, while avoiding the use of gelatin.

WO 2017/112763 discloses a high integrity encapsulation product with insoluble fibers, wherein an agent to be encapsulated is encapsulated in a glassy matrix. The glassy matrix contains at least one modified starch, at least one low molecular weight carbohydrate, and at least one insoluble fiber (0.5-10%).

Such particles, which may also contain other active ingredients or functional components such as flavoring or odiferous substances, are added, for example, to gel-based toothpastes or skin creams to improve their visual appearance, impart a texture or provide aroma.

However, the prior art particles have the disadvantage that they are unstable in an aqueous matrix, such as a gel toothpaste, a food product, such as a yogurt product, or cleaning products, when stored for a long time and tend to bleed out or even dissolve. Another disadvantage of prior art color particles is that they may be perceived by the consumer as foreign bodies when used.

Furthermore, the processing of carbohydrate matrices is limited due to the viscosity of their melt, especially during melt extrusion. In the case of matrices with a high viscosity of the melt, flow through small openings in the extruder is no longer possible, while in the case of matrices with a low viscosity, the melt is too soft and the particles formed tend to stick together. Some carbohydrate matrices are already ruled out for use as color particles because of their natural coloration: For example, corn starch naturally exhibits a beige hue that is less suitable for optical purposes than a pure white hue.

There is thus a growing demand for particles in a wide variety of applications such as foods, beverages, cosmetics, especially oral hygiene products, pharmaceuticals and consumer goods, especially detergents and cleaning agents in liquid, gel or powder form, pet food or pet care products, to improve their appearance or consistency. In general, maximum stability or shelf life on the one hand and high producibility on the other hand of these particles are sought.

The task of the present invention was to provide color particles that are stable over an extended storage time, particularly in an aqueous matrix, i.e., do not bleed out or dissolve, are not perceived as objectionable in use, and have a clean colored, including white, matrix.

Furthermore, it was an aim of the present invention to provide color particles that can be produced with high producibility by extrusion. Especially for use in toothpastes, dairy products, preferably yogurt products, or cleaning products, preferably liquid soap or toilet cleaner, the color particle matrix must have sufficient strength for the manufacturing process and at the same time, however, it must be soft enough when used, for example when consumed, so as not to be perceived as disturbing.

In addition, the color particles should be available from bio-based or sustainably produced raw materials.

### SUMMARY OF THE INVENTION

The present problem is solved by the objects of the independent patent claims. Preferred embodiments result from the wording of the dependent patent claims, the following description and the examples.

In a first aspect, the present invention relates to color particles comprising or consisting of an extrudate from an extrusion process, wherein the extrudate comprises or consists of:
a carbohydrate matrix comprising or consisting of a starch having an amylopectin content of at least 80% and at least one viscosity modifying component;
at least one/one emulsifier;
water; and
optionally at least one colorant and/or at least one flavoring or odiferous substance or at least one aroma or scent.

The second object of the present invention is a method for producing a color particle, comprising the steps of:
(i) providing a combination of a carbohydrate matrix comprising or consisting of a starch having an amylopectin content of at least 80% and at least one viscosity modifying component;
(ii) preparation of a mixture of the combination obtained from step (i) and at least one emulsifier, water and optionally at least one colorant and/or at least one flavoring or odiferous substance and/or at least one aroma or scent to obtain a suspension or gel;
(iii) extrusion of the suspension or gel to obtain an extrudate;
(iv) comminution of the extrudate to obtain a color particle; and
(v) optional drying of the color particle.

In a third aspect, the present invention relates to color particles, obtainable according to the process of the invention.

A further object of the present invention is the use of the color particles according to the invention for the production of foodstuffs, beverages, cosmetics, in particular oral hygiene products, pharmaceuticals, consumer goods, in particular detergents and cleaning agents in liquid, gel or powder form, pet food or pet care products.

Ultimately, the present invention relates to foodstuffs, beverages, cosmetics, in particular oral hygiene products, pharmaceuticals, consumer articles, in particular detergents and cleaning agents in liquid, gel or powder form, pet food or pet care products comprising the color particles according to the invention.

### DESCRIPTION OF THE FIGURES

FIG. 1 is an illustration showing the stability of color particles prepared according to Examples 5 and 8 in different toothpaste bases (silica base (prepared according to Example 2); carbonate base (prepared according to Example 3); and gel base (prepared according to Example 3) after storage for 3 weeks at 45° C.

### DETAILED DESCRIPTION OF THE INVENTION

In a first aspect, the present invention relates to color particles comprising or consisting of an extrudate from an extrusion process, wherein the extrudate comprises or consists of:
a carbohydrate matrix comprising or consisting of a starch having an amylopectin content of at least 80% and at least one viscosity modifying component;
at least one/one emulsifier;
water; and
optionally at least one colorant and/or at least one flavoring or odiferous substance or at least one aroma or scent.

The color particles according to the invention comprise or consist of an extrudate. This extrudate is prepared from a mixture or combination of a carbohydrate matrix comprising or consisting of a starch with an amylopectin content of at least 80% and at least one viscosity modifying component. The two aforementioned components of the carbohydrate matrix form the body of the color particles and are also carriers for other, optional components that can be added to the color particles.

Starch is a polysaccharide with the formula (C6 Hio 05)n, which consists of α-D-glucose-units linked by glycosidic bonds. The macromolecule is therefore classified as a carbohydrate. Starch is one of the most important reserve substances in plant cells.

Particularly rich in starch, and therefore used for the extraction of isolated starch, are:
Corn
Wheat
Rice
Potatoes
Cassava (Tapioca)

Starch consists mostly of
1-30% amylose, linear chains with helical (screw) structure linked only α-1,4-glycosidically; and
70-99% amylopectin, highly branched structures, with α-1,6-glycosidic and α-1,4-glycosidic linkages. Starch amylopectin is branched with about one α-1,6-glycosidic linkage per about 30 α-1,4-glycosidic linkages.

The proportions of amylose and amylopectin differ depending on the starch source.

Table 1 below shows the composition of starches (amylose content) and their solubility (from “Lehrbuch der Lebensmittelchemie”, ed.: Belitz, Grosch, Schieberle, 5th completely revised edition, 2001, Springer Publisher):

TABLE 1

| | Amylose content | Solubility at 80° C. |
|---|---|---|
| Strength | (%) | (%) |
| Wheat | 26-31 | 2.59 |
| Rye | 28 | |
| Barley | 22-29 | |
| Corn | 28 | |
| Wax corn | 0-1 | |
| Oats | 27 | |
| Rice | 14-32 | |
| Potato | 23 | 31.0 |
| Cassava | 17 | |

Due to the different composition, the above-mentioned starches have different physical and chemical properties.

Starch is used in a variety of ways, either as native or modified starch. Modified starches are starch products obtained by physical, chemical or enzymatic processes that meet increased technological requirements. The grain structure and other essential properties are retained after modification.

Modified starches are used in the food industry because, compared to natural starch, they have better
Heat stability
Acid stability
Shear stability, as well as
freezing and thawing behavior

However, such strengths require procedural pretreatment.

In the context of the present invention and the following description, the term “starch” is understood to include both native starch(es) and modified starch(es), i.e., starch products obtained by physical, chemical or enzymatic processes, from different starch sources.

Surprisingly, it has now been found that a starch with an amylopectin content of at least 80% is particularly well suited for producing color particles with high producibility by extrusion processes, which are stable over a longer storage time, especially in an aqueous matrix, i.e., do not bleed out or dissolve, are not perceived as a disturbing foreign body when used, and, in addition, have a clean colored, including white, matrix.

According to the invention, a starch having an amylopectin content of at least 80% is preferably used for the production of the color particles according to the invention.

In a preferred embodiment of the color particles according to the invention, the starch having an amylopectin content of at least 80% is selected from the group consisting of rice starch, potato starch and combinations of the two mentioned starches.

Most preferred is starch with an amylopectin content of at least 90%.

The most suitable is amylopectin potato starch for the formation of the carbohydrate matrix. Amylopectin potato starch is based on natural breeding and has an amylopectin content of at least 95% and contains virtually no amylose. Such an amylopectin potato starch is commercially available, for example, under the name “Eliane™ Gel 100”. Amylopectin potato starch is characterized by high purity, particular process stability and very high viscosity.

In a preferred embodiment, the starch has a viscosity of 10 mPa·s to 50.000 mPa·s, preferably a viscosity of 20 mPa·s to 30.000 mPa·s, measured as a 10% solution in water at 90° C. using a rheometer (Anton Paar Rheometer MCR302, cone-plate system, cone type CP-50-1 at a constant shear rate of 5 sec⁻¹).

Most preferred for the preparation of the color particles according to the present invention are starches from potatoes and rice having a viscosity of 10 mPa·s to 50.000 mPa·s, preferably a viscosity of 20 mPa·s to 30.000 mPa·s, measured as a 10% solution in water at 90° C. using a rheometer (Anton Paar Rheometer MCR302, cone-plate system, cone type CP-50-1 at a constant shear rate of 5 sec¹).

In addition, the amylopectin potato starch has the following properties:
smooth and glossy appearance, high clarity and creamy texture;
high viscosity, high elasticity and high water-binding capacity;
processable at low temperatures;
neutral in taste and odor and good flavor acceptance;
high temperature stability and resistance to shear forces.

Amylopectin potato starch has been used primarily as a thickener or in dry applications, for example in instant products such as soups and sauces, but not in an aqueous matrix, such as in toothpastes, dairy products, especially yogurt, or cleaning agents.

The starches used according to the invention have melting points above 170° C., preferably above 180° C. The carbohydrate polymers used have glass transition temperatures above 70° C., preferably above 80° C., in particular preferably above 90° C. (glass transition temperatures determined by differential scanning calorimetry (DSC 200 F3 Netzsch)).

The further at least one viscosity-modifying constituent of the carbohydrate matrix of the color particles according to the invention is a water-soluble compound selected from the group consisting of sugars, sugar alcohols, dextrins or maltodextrins, and mixtures of the aforementioned compounds.

Particularly suitable and preferred sugars are mono, di and trisaccharides, which in turn are selected from the group consisting of, for example, arabinose, xylose, fructose, galactose, glucose, mannose, sorbose, lactose, maltose, sucrose or maltotriose.

Sugar alcohols as a water-soluble compound are particularly preferred in the production of the color particles according to the invention.

The sugar alcohol used according to the invention is preferably selected from the group consisting of sorbitol, mannitol, isomalt, lactitol, xylitol, threitol, erythritol, arabinol, arabitol, adontol, alditol, ducitol, iditol and mixtures of the aforementioned sugar alcohols.

Particularly preferably, sorbitol is used to prepare the carbohydrate matrix.

If isomers of the previously mentioned compounds exist, either the pure isomers or any mixtures thereof can be used.

The water-soluble compounds facilitate granulation at the perforated nozzles. On the other hand, the water-soluble compounds act advantageously as humectants to prevent the color particles of the invention from drying out. The compounds interact with the starch contained, which means that the starch retains its water-binding capacity for longer and the color particles therefore dry out less quickly.

The amount of the carbohydrate matrix comprising or consisting of a starch having an amylopectin content of at least 80% and at least one viscosity modifying ingredient is from 50% to 98% by weight based on the total weight of the color particle. Preferably, the carbohydrate matrix is present in the color particle in an amount of from 60 to 98% by weight and most preferably in an amount of from 80 to 98% by weight, based on the total weight of the color particle.

In another preferred embodiment according to the first aspect of the present invention, the ratio of starch having an amylopectin content of at least 80% to the viscosity modifying ingredient ranges from 99:1 to 50:50.

The color particle according to the invention further comprises an emulsifier as a further component. Advantageously, a small amount of at least one emulsifier is incorporated in order to increase the solubility or the emulsifiability or the suspendability of the constituents when preparing the color particle according to the invention. The addition of the at least one emulsifier also simultaneously increases the stability of the suspension or gel obtained. In addition, the at least one emulsifier in the carbohydrate matrix improves the transport of the material melt during extrusion, reduces the adhesion of the material melt to the walls of the extruder, and improves the flow of material through the dies of the extruder.

Suitable and preferred emulsifiers are emulsifiers selected from the group consisting of monoglycerides, diglycerides, deca-glycerol dipalmitate, hexa-glycerol distearate, polyglycerol esters, sulfoacetates, lecithin, polysorbates and mixtures of said emulsifiers.

Monoglycerides, diglycerides or lecithin are particularly preferred emulsifiers.

The at least one emulsifier is added to the color particle according to the invention in an amount of 0.1 to 5% by weight, based on the total weight of the color particle. Preferably, the emulsifier is present in the color particle in an amount of 0.2 to 4 wt. % and most preferably in an amount of 0.5 to 3 wt. %, based on the total weight of the color particle.

As a further component, the color particles according to the invention comprise water, which is necessary to be able to produce a viscous mixture in the form of a suspension or a gel from the above-mentioned components. Depending on the starting ingredients, in particular the starch used, a suspension or gel is formed after mixing together the ingredients of the color particles according to the invention and after adding the emulsifier. When the starch slurry is heated or warmed up, a gel is preferably formed.

In addition, the water content ensures that this mixture is still flowable enough to be dispensed through small openings at acceptable temperatures of <130° C. and not too high extrusion pressure of <60 bar.

Typically, the water content of the color particles according to the invention is 1 to 30% by weight, based on the total weight of the color particle.

A water content that is too low makes processing difficult due to the high viscosity of the compound, so that flow through small openings is no longer possible. A water content that is too high, on the other hand, results in a compound that is too soft and cannot be processed via extrusion or can only be processed very poorly.

Preferably, the water content of the color particles according to the invention is 1 to 20% by weight, even more preferably 5 to 15% by weight, but most preferably 30% by weight, based on the total weight of the color particle.

The color particles according to the invention may optionally contain, in addition to the above-mentioned ingredients, one or more further (customary) active ingredients or functional ingredients, which make up the sum to 100% by weight in the color particles according to the invention.

Compared to other starches, for example corn starch, the carbohydrate matrix advantageously has a pure white color, which already makes it suitable as such as color particles, for example as color particles in toothpastes, gels, dairy products, for example yogurt, or cleaning agents.

For optical purposes, at least one further colorant may optionally be added to the color particle according to the invention. The at least one colorant is a colorant or pigment colorant selected from the group consisting of: E100, E101, E102, E104, E110, E120, E122, E123, E124, E127, E128, E129, E131, E132, E133, E141, E141(i), E141(ii), E142, E150a, E150b, E150c, E150d, E151, E153, E155, E160, E160a, E160b, E160c, E161b, E163, E171, E172, E174, E175 and mixtures of the above colorants or pigment colorants.

Other colorants or pigment colorants used and added to the color particles according to the invention are titanium dioxide E171 C.I. 77891, pearlescent pigment silver AA, gold E175, blue C.I. 74160, iron oxide red E172 C.I. 77491, pearlescent pigment gold BB, vegetable carbon E153 C.I. 77268:1, red C.I. 73360, green C.I. 74260, iron oxide black E172 C.I. 77499 or mixtures of the above colorants and pigment colorants.

Particularly preferred are oil-soluble colorants and pigment colorants selected from the group consisting of: E141, E153, E160, E 160a, E 160b, E 160c, E171, E172, CI11680, CI12085, CI12490, CI13015, CI15850, CI16185, CI18965, CI19140, CI42045, CI42051, CI42090, CI45350, CI45410, CI47005, CI59040, CI60725, CI61565 CI61570, CI74160, CI74260, CI77007, CI77019, CI77266, CI73360, CI77492, CI77499, CI77891 and mixtures of the abovementioned colorants or pigment colorants.

The foregoing lists are illustrative and not intended to be exhaustive.

The previously mentioned colorants are in particular oil-soluble colorants and pigment colorants approved for food, beverages, oral care products and cosmetics. Water-soluble colorants are less suitable because they tend to bleed out in an aqueous matrix, for example in a toothpaste, a gel, a dairy product, preferably a yogurt, or a cleaning product.

The at least one colorant is added to the color particle of the invention in an amount of 0.001 to 10% by weight, based on the total weight of the color particle. Preferably, the at least one colorant is included in the color particle in an amount of 0.01 to 2 wt % and most preferably in an amount of 0.1 to 1 wt %, based on the total weight of the color particle.

The color particles according to the invention optionally contain as a further component at least one flavoring or at least one aroma, i.e., two, three, four, five or even far more aroma components in a sensory effective amount, or at least one further odiferous substance or at least one scent or two, three, four, five or even far more odiferous substance components in a sensory effective amount. The terms “flavoring” and “aroma” or “odiferous substance” and “scent” are used in the context of the present application equally next to each other and are therefore dependent on the intended use, namely flavoring on the one hand or fragrancing or perfuming on the other hand.

The term “sensory effective amount” in the context of the present application means that the flavoring or aroma or odiferous substance or scent is used in such a sufficient amount that the resulting product, in operation or in use, reveals the sensory properties of the flavoring or aroma or odiferous substance or scent.

Typically, flavoring or odiferous compounds are not used in binary or ternary blends, but as components of sophisticated complex blends that may contain two, three, four, five, ten, or preferably even a much higher number of flavoring or odiferous compounds, in some cases in very small amounts, to give a particularly rounded flavor profile.

In a preferred further development of the present invention, the color particles therefore optionally contain any desired number of further flavoring or odiferous compounds selected from the group formed by: (1) hydrocarbons; (2) aliphatic alcohols; (3) aliphatic aldehydes and their acetals; (4) aliphatic ketones and their oximes; (5) aliphatic sulfur-containing compounds; (6) aliphatic nitriles; (7) esters of aliphatic carboxylic acids; (8) acyclic terpene alcohols; (9) acyclic terpene aldehydes and ketones; (10) cyclic terpene alcohols; (11) cyclic terpene aldehydes and ketones; (12) cyclic alcohols; (13) cycloaliphatic alcohols; (14) cyclic and cycloaliphatic ethers; (15) cyclic and macrocyclic ketones; (16) cycloaliphatic aldehydes; (17) cycloaliphatic ketones; (18) esters of cyclic alcohols; (19) esters of cycloaliphatic alcohols; (20) esters of cycloaliphatic carboxylic acids; (21) araliphatic alcohols; (22) esters of araliphatic alcohols and aliphatic carboxylic acids; (23) araliphatic ethers; (24) aromatic and araliphatic aldehydes; (25) aromatic and araliphatic ketones; (26) aromatic and araliphatic carboxylic acids and esters thereof; (27) nitrogen-containing aromatic compounds; (28) phenols, phenyl ethers and phenyl esters; (29) heterocyclic compounds; (30) lactones; and mixtures thereof.

The selection of flavorings or odiferous substances is very comprehensive in this respect; corresponding substances with which the color particles according to the invention can be advantageously combined can be found, for example, in “S. Arctander, Perfume and Flavor Chemicals, Volumes I and II, Montclair, N.J., 1969, self-published” or “H. Surburg and J. Panten, Common Fragrance and Flavor Materials, 6th edition, Wiley-VCH, Weinheim, 2016”.

In detail, the following may be mentioned:

Extracts from natural raw materials: This group represents essential oils, concretes, absolutes, resins, resinoids, balsams, tinctures such as Ambergris tincture; Amyris oil; Angelica seed oil; Angelica root oil; Anise oil; Valerian oil; Basil oil; Tree moss absolute; Bay oil; Mugwort oil; Benzoeresin; Bergamot oil; Beeswax absolute; Birch tar oil; Bitter almond oil; Savory oil; Bucco leaf oil; Cabreuva oil; Cade oil; Calmus oil; Camphor oil; Cananga oil; Cardamom oil; Cascarilla oil; Cassia oil; Cassie-absolute; Castoreum-absolute; Cedar leaf oil; Cedarwood oil; Cistus oil; Citronella oil; Citron oil; Copaiva balsam; Copaiva balsam oil; Coriander oil; Costus root oil; Cumin oil; Cypress oil; Davana oil; Dill herb oil; Dill seed oil; Eau de brouts absolute; Oak moss absolute; Elemi oil; Tarragon oil; Eucalyptus citriodora oil; Eucalyptus oil; Fennel oil; Spruce needle oil; Galbanum oil; Galbanum resin; Geranium oil; Grapefruit oil; Guaiac wood oil; Gurjun balsam; Gurjun balsam oil; Helichrysum absolute; Helichrysum oil; Ginger oil; Iris root absolute; Iris root oil; Jasmine absolute; Calamus oil; Chamomile oil blue; Chamomile oil Roman; Carrot seed oil; Cascarilla oil; Pine needle oil; Curly mint oil; Caraway seed oil; Labdanum oil; Labdanum absolute; Labdanum resin; Lavandin absolute; Lavandin oil; Lavender absolute; Lavender oil; Lemongrass oil; Lovage oil; Lime oil distilled; Lime oil pressed; Linaloe oil; Litsea cubeba oil; Bay leaf oil; Mace oil; Marjoram oil; Mandarin oil; Masso bark oil; Mimosa absolute; Musk grain oil; Musk tincture; Muscat oil; Myrrh absolute; Myrrh oil; Myrtle oil; Clove leaf oil; Clove flower oil; Neroli oil; Olibanum absolute; Olibanum oil; Opopanax oil; Orange flower absolute; Orange oil; Origanum oil; Palmarosa oil; Patchouli oil; Perilla oil; Perubalsam oil; Parsley leaf oil; Parsley seed oil; Petitgrain oil; Peppermint oil; Pepper oil; Allspice oil; Pine oil; Poley oil; Rose absolute; Rosewood oil; Rose oil; Rosemary oil; Sage oil Dalmatian; Sage oil Spanish; Sandalwood oil; Celery seed oil; Spicy lavender oil; Star anise oil; Styrax oil; Tagetes oil; Fir needle oil; Tea tree oil; Turpentine oil; Thyme oil; Tolu balsam; Tonka absolute; Tuberose absolute; Vanilla extract; Violet leaf absolute; Verbena oil; Vetiver oil; Juniper berry oil; Wine yeast oil; Wormwood oil; Wintergreen oil; Ylang oil; Hyssop oil; Civet absolute; Cinnamon leaf oil; Cinnamon bark oil, and fractions thereof, or ingredients isolated therefrom.

Individual odiferous substances and flavorings: Individual odiferous substances and flavorings can be divided into a variety of classes, namely:

hydrocarbons, such as 3-carene; α-pinene; β-pinene; α-terpinene; γ-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane;

aliphatic alcohols such as hexanol; octanol; 3-octanol; 2,6-dimethylheptanol; 2-methyl-2-heptanol; 2-methyl-2-octanol; (E)-2-hexenol; (E) and (Z)-3-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;

aliphatic aldehydes and their acetals such as hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal;2,6,10-trimethyl-9-undecenal; 2,6,10-trimethyl-5,9-undecadienal; heptanal diethyl acetal; 1,1-dimethoxy-2,2,5-trimethyl-4-hexene; citronellyloxyacetaldehyde; 1-(1-methoxy-propoxy)-(E/Z)-3-hexene;

aliphatic ketones and their oximes such as 2-heptanone; 2-octanone; 3-octanone; 2-nonanone; 5-methyl-3-heptanone; 5-methyl-3-heptanonoxime; 2,4,4,7-tetramethyl-6-octen-3-one; 6-methyl-5-hepten-2-one;

aliphatic sulfur-containing compounds such as 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthen-8-thiol;

aliphatic nitriles such as 2-nonenoic acid nitrile; 2-undecenoic acid nitrile; 2-tridecenoic acid nitrile; 3,12-tridecadienoic acid nitrile; 3,7-dimethyl-2,6-octadienoic acid nitrile; 3,7-dimethyl-6-octenoic acid nitrile;

esters of aliphatic carboxylic acids such as (E) and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E) and (Z)-3-hexenyl acetate; octyl acetate; 3-octyl acetate; 1-octen-3-yl acetate; ethyl butyrate; butyl butyrate; isoamyl butyrate; hexyl butyrate; (E) and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; ethyl (E,Z)-2,4-decadienoate; methyl 2-octinate; methyl 2-noninate; allyl 2-isoamyloxyacetate; methyl 3,7-dimethyl-2,6-octadienoate;4-methyl-2-pentyl crotonate;

acyclic terpene alcohols such as citronellol; geraniol; nerol; linalool; lavadulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and their formates, acetates, propionates, isobutyrates, butyrates, isovalerianates, pentanoates, hexanoates, crotonates, tiglinates and 3-methyl-2-butenoates;

acyclic terpene aldehydes and ketones such as geranial; neral; citronellal; 7-hydroxy-3,7-dimethyloctanal; 7-methoxy-3,7-dimethyloctanal; 2,6,10-trimethyl-9-undecenal; geranylacetone; and the dimethyl and diethylacetals of geranial, neral, 7-hydroxy-3,7-dimethyloctanal;

cyclic terpene alcohols such as menthol; isopulegol; alpha-terpineol; terpinenol-4; menthan-8-ol; menthan-1-ol; menthan-7-ol; borneol; isoborneol; linalool oxide; nopol; cedrol; ambrinol; vetiverol; guaiaol; and their formates, acetates, propionates, isobutyrates, butyrates, isovalerianates, pentanoates, hexanoates, crotonates, tiglinates and 3-methyl-2-butenoates;

cyclic terpene aldehydes and ketones such as menthone; isomenthone; 8-mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-on; 1,3,4,6,7,8a-hexahydro-1,1,5,5-tetramethyl-2H-2,4a-methanonaphthalen-8(5H)-on; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedarwood oil (methylcedrylketone);

cyclic alcohols such as 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;

cycloaliphatic alcohols such as alpha,3,3-trimethylcyclohexyl-methanol;1-(4-isopropylcyclohexyl)ethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;

cyclic and cycloaliphatic ethers such as cineol; cedryl methyl ether; cyclododecyl methyl ether; 1,1-dimethoxycyclododecane; (ethoxymethoxy)cyclodo-decane; alpha-cedrene epoxide; 3a,6,6,9a-tetramethyldodecahydronaphtho[2,1-b]furan; 3a-ethyl-6,6,9a-trimethyldodecahydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropyl)-1,3-dioxane;

cyclic and macrocyclic ketones such as 4-tert.butylcyclohexanone; 2,2,5-trimethyl-5-pentylcyclopentanone; 2-heptylcyclopentanone; 2-pentylcyclo-pentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopenta-decenone; 3-methyl-5-cyclopentadecenone; 3-methylcyclopentadecanone; 4-(1-ethoxyvinyl)-3,3,5,5-tetramethylcyclohexanone; 4-tert.-pentylcyclohexanone; 5-cyclohexadecen-1-one; 6,7-dihydro-1,1,2,3,3-pentamethyl-4(5H)-indanone; 8-cyclohexadecen-1-one; 9-cycloheptadecen-1-one; cyclopentadecanone; cyclohexa-decanone;

cycloaliphatic aldehydes such as 2,4-dimethyl-3-cyclohexenecarbaldehyde; 2-methyl-4-(2,2,6-trimethyl-cyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;

cycloaliphatic ketones such as 1-(3,3-dimethylcyclohexyl)-4-penten-1-on; 2,2-dimethyl-1-(2,4-dimethyl-3-cyclohexen-1-yl)-1-propanon; 1-(5,5-dimethyl-1-cyclohexen-1-yl)-4-penten-1-on; 2,3,8,8-tetramethyl-1,2,3,4,5,6,7,8-octahydro-2-naphthalenyl methyl ketone; methyl 2,6,10-trimethyl 2,5,9-cyclododecatrienyl ketone; tert.-butyl-(2,4-dimethyl-3-cyclohexen-1-yl)ketone;

esters of cyclic alcohols such as 2-tert-butyl cyclohexyl acetate; 4-tert-butyl cyclohexyl acetate; 2-tert-pentyl cyclohexyl acetate; 4-tert-pentyl cyclohexyl acetate; 3,3,5-trimethyl cyclohexyl acetate; decahydro-2-naphthyl acetate; 2-cyclopentyl cyclopentyl crotonate; 3-pentyl tetrahydro-2H-pyran-4-yl acetate; decahydro-2,5,5,8a-tetramethyl-2-naphthyl acetate; 4,7-methano-3a,4,5,6,7,7a-hexahydro-5, respectively 6-indenyl acetate; 4,7-methano-3a,4,5,6,7,7a-hexahydro-5, or 6-indenyl propionate; 4,7-methano-3a,4,5,6,7,7a-hexahydro-5, or 6-indenyl isobutyrate; 4,7-methanooctahydro-5, or 6-indenyl acetate;

esters of cycloaliphatic alcohols such as 1-cyclohexylethyl crotonate;

esters of cycloaliphatic carboxylic acids such as allyl 3-cyclohexyl propionate; allyl cyclohexyloxy acetate; cis and trans-methyl dihydrojasmonate; cis and trans-methyl jasmonate; methyl 2-hexyl-3-oxocyclopentane carboxylate; ethyl-2-ethyl-6,6-dimethyl-2-cyclohexenecarboxylate; ethyl-2,3,6,6-tetramethyl-2-cyclohexenecarboxylate; ethyl 2-methyl-1,3-dioxolane-2-acetate;

araliphatic alcohols such as benzyl alcohol; 1-phenylethyl alcohol; 2-phenylethyl alcohol; 3-phenyl propanol; 2-phenyl propanol; 2-phenoxyethanol; 2,2-dimethyl-3-phenyl propanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;

esters of araliphatic alcohols and aliphatic carboxylic acids such as benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethyl benzyl acetate; alpha,alpha-dimethylphenyl ethyl acetate; alpha,alpha-dimethylphenyl ethyl butyrate; cinnamyl acetate; 2-phenoxyethyl isobutyrate; 4-methoxybenzyl acetate;

araliphatic ethers such as 2-phenylethyl methyl ether; 2-phenylethyl isoamyl ether; 2-phenylethyl 1-ethoxyethyl ether; phenylacetaldehyde dimethyl acetal; phenylacetaldehyde dimethyl acetal; hydratropaaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;

aromatic and araliphatic aldehydes such as benzaldehyde; phenylacetaldehyde; 3-phenylpropanal; hydratropaaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert.-butylphenyl)propanal; 2-methyl-3-(4-isobutylphenyl)propanal; 3-(4-tert.-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;

aromatic and araliphatic ketones such as acetophenone; 4-methylacetophenone; 4-methoxyacetophenone; 4-tert.butyl-2,6-dimethylacetophenone; 4-phenyl-2-butanone; 4-(4-hydroxyphenyl)-2-butanone; 1-(2-naphthalenyl)ethanone; 2-benzofuranylethanone; (3-methyl-2-benzofuranyl)ethanone; benzophenone; 1,1,2,3,6-hexamethyl-5-indanylmethyl ketone; 6-tert.butyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′,8′-tetrahydro-3′,5′, 5′,6′,8′,8′-hexamethyl-2-acetonaphthone;

aromatic and araliphatic carboxylic acids and their esters such as benzoic acid; phenylacetic acid; methyl benzoate; ethyl benzoate; hexyl benzoate; benzyl benzoate; methyl phenyl acetate; ethyl phenyl acetate; geranyl phenyl acetate; phenyl ethyl phenyl acetate; methyl cinnamate; ethyl cinnamate; benzyl cinnamate; phenyl ethyl cinnamate; cinnamyl cinnamate; allyl phenoxy acetate; methyl salicylate; isoamyl salicylate; hexyl salicylate; cyclohexyl salicylate; cis-3-hexenyl salicylate; benzyl salicylate; phenyl ethyl salicylate; methyl 2,4-dihydroxy-3,6-dimethylbenzoate; ethyl 3-phenyl glycidate; ethyl 3-methyl-3-phenyl glycidate;

nitrogen-containing aromatic compounds such as 2,4,6-trinitro-1,3-dimethyl-5-tert-butylbenzene; 3,5-dinitro-2,6-dimethyl-4-tert.butylacetophenone; cinnamic acid nitrile; 3-methyl-5-phenyl-2-pentenoic acid nitrile; 3-methyl-5-phenylpentanoic acid nitrile; methyl anthranilate; methyl-N-methyl anthranilate; Schiff'sche bases of methyl anthranilate with 7-hydroxy-3,7-dimethyloctanal, 2-methyl-3-(4-tert.-butylphenyl)propanal or 2,4-dimethyl-3-cyclohexenecarbaldehyde; 6-isopropylquinoline; 6-isobutylquinoline; 6-sec-butylquinoline;2-(3-phenylpropyl)pyridine; indole; scatole; 2-methoxy-3-isopropylpyrazine; 2-isobutyl-3-methoxypyrazine;

phenols, phenyl ethers and phenyl esters such as tarragol; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresylphenyl acetate;

heterocyclic compounds such as 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;

lactones such as 1,4-octanolide; 3-methyl-1,4-octanolide; 1,4-nonanolide; 1,4-decanolide; 8-decene-1,4-olide; 1,4-undecanolide; 1,4-dodecanolide; 1,5-decanolide; 1,5-dodecanolide;4-methyl-1,4-decanolide; 1,15-pentadecanolide; cis and trans-11-pentadecene-1,15-olide; cis and trans-12-pentadecene-1,15-olide; 1,16-hexadecanolide; 9-hexadecene-1,16-olide; 10-oxa-1,16-hexadecanolide; 11-oxa-1,16-hexadecanolide; 12-oxa-1,16-hexadecanolide; ethylene-1,12-dodecanedioate; ethylene-1,13-tridecanedioate; coumarin; 2,3-dihydrocoumarin; octahydrocoumarin;

as well as any mixtures of the aforementioned flavoring or odiferous substances.

Flavorings or odiferous substances or aromas can be used in liquid form undiluted or diluted with a solvent for aromatization. Suitable and preferred solvents for this purpose are in particular ethanol, glycerol, vegetable oil, triglycerides, 1,2-propylene glycol, 1,2-butylene glycol, dipropylene glycol, diethyl phthalate, triethyl citrate, isopropyl myristate and triacetin.

Such flavoring or odiferous substance mixtures contain up to 99% by weight, preferably about 5% to about 70% by weight, in particular about 10% to about 50% by weight and particularly preferably about 15% to about 25% by weight of said solvents.

In a preferred alternative, the flavoring or odiferous substance mixtures comprise synthetic or natural, preferably tasteless and odorless carrier substances, in particular carrier oils, which contain the flavoring or odiferous substances in highly concentrated form and optionally solvents and/or excipients.

For some applications, it is also advantageous to adsorb the flavoring or odiferous substance mixtures to a carrier that provides both a fine distribution of the flavorings or odiferous substances contained therein in the product and a controlled release upon application. Such carriers can be porous inorganic materials such as light sulfate, silica gels, zeolites, gypsums, clays, clay granules, aerated concrete, etc., or organic materials such as woods, cellulose-based materials, sugars, dextrins (e.g., maltodextrin) or cyclodextrins.

In an alternative preferred embodiment, the flavoring or odiferous substance mixtures are in microencapsulated or spray-dried form or are present as inclusion complexes or as extrusion products to be added in this form to the flavored product.

The microencapsulation of the flavoring or odiferous substance compositions can be carried out, for example, by the so-called coacervation process using capsule materials, e.g., made of soft gelatin. The spray-dried scent, aroma or odiferous substance compositions can be prepared, for example, by spray-drying an emulsion or dispersion containing the flavoring or odiferous substance mixture according to the invention, wherein modified starches, proteins, dextrin and vegetable gums can preferably be used as carriers. Inclusion complexes can be prepared, for example, by incorporating dispersions of a flavoring or odiferous substance blend and cyclodextrins or urea derivatives into a suitable solvent, such as water. Extrusion products can be obtained by fusing a flavoring or odiferous substance blend with a suitable waxy material and by extrusion followed by solidification, optionally in a suitable solvent, e.g., isopropanol.

If necessary, the properties of flavoring or odiferous substance mixture preparations modified in this way can be further optimized by so-called “coating” with suitable materials with a view to more targeted scent release.

The color particles according to the invention have a flavoring, aroma or odiferous substance or scent content of from 0.01 to 25% by weight, preferably from 0.1 to 15% by weight, more preferably from 0.2 to 10% by weight, based on the total weight of the color particle.

Optionally, the color particles according to the invention comprise further active ingredients or functional ingredients selected from the group consisting of: preservatives, antioxidants, UV filters, vitamins, acidity regulators, sweeteners, stabilizers, thickeners, gelling agents, anticaking agents, and other ingredients commonly used and approved in the food, cosmetics and pharmaceutical industries.

In a particularly preferred variant of the first aspect, the color particles according to the invention have the following composition:
50 to 98% by weight of a carbohydrate matrix of a starch having an amylopectin content of at least 80% and at least one viscosity modifying component;
0.1 to 5% by weight of at least one emulsifier;
1 to 30% by weight of water; and
optionally 0.001 to 10% by weight of at least one colorant;
optionally 0.01 to 25% by weight of at least one flavoring or odiferous substance or at least one aroma or scent;
   based on the total weight of the color particle.

The color particles according to the invention typically have a glass transition temperature in the range of 10 to 90° C., preferably in the range of 20 to 75° C., particularly preferably in the range of 20 to 60° C. The glass transition temperatures were determined by means of differential scanning calorimetry (DSC 200 F3 Netzsch).

The color particles according to the first aspect of the present invention preferably have a cylindrical or spherical geometry and a narrow particle size profile. They typically have a diameter of from 0.2 to 5 mm, preferably from 0.3 to 3.0 mm, and more preferably from 0.5 to 2.0 mm, and/or a length of from 0.1 to 10 mm, preferably from 0.2 to 3.0 mm, and more preferably from 0.3 to 1.5 mm. The aforementioned sizes thus preferably lie in the range of 0.1 to 1.5 mm in which particles are optically perceived.

As a comparison of the color particles according to the invention produced from rice or potato starch with color particles produced from corn, wheat or tapioca starch shows, the color particles according to the invention are characterized by good producibility in the extruder as well as good storage stability over a longer period of time. Even in a moist matrix, the color particles according to the invention do not bleed out or dissolve. Due to these properties, the color particles according to the invention are particularly suitable for improving the optical appearance of gel-based preparations, such as toothpastes or skin care products, cleaning products or dairy products, such as yogurt, or for imparting texture thereto, especially over the shelf life of such products.

The color particles of the invention are also readily friable in water, which makes them eminently suitable for the intended applications, for example in toothpastes, dairy products such as yogurt or cleaning products. The good stability and friability of the color particles in water is a measure of the fact that the color particles are not perceived as annoying or as rough when used. In addition, the color particles of the invention have a clean colored, including white, matrix.

The effects described above are illustrated by the results in Tables 2 and 3 below.

A further advantage of the color particles according to the invention is an optionally high loadability with flavorings or odiferous substances or aromas or scents, extracts or active ingredients as well as a maximum retention of flavorings or odiferous substances or aromas or scents, or extracts or active ingredients during their storage or over the shelf life of the end products.

The second object of the present invention is a method for producing a color particle, comprising the steps of:
(i) providing a combination of a carbohydrate matrix comprising or consisting of a starch having an amylopectin content of at least 80% and at least one viscosity modifying component;
(ii) preparation of a mixture of the combination obtained from step (i) and at least one emulsifier, water and optionally at least one colorant and/or at least one flavoring or odiferous substance and/or at least one aroma or scent to obtain a suspension or gel;
(iii) extrusion of the suspension or gel to obtain an extrudate;
(iv) comminution of the extrudate to obtain a color particle; and
(v) optional drying of the color particle.

The color particles according to the invention are produced by extrusion in an extruder. After mixing and dispersing or suspending the components of the color particles according to the invention, the highly viscous mixture, i.e., the suspension or gel, is continuously pressed or extruded through the die(s) of the extruder and comminuted in a subsequent step.

The preferred extruder type used is a twin-screw extruder, although other known extruder types can also be used. Preferably, a twin-screw extruder is used for mixing the components of the color particle according to the invention, which can be equipped with several temperature zones so that the temperature during mixing and extrusion can be controlled in a targeted manner.

In a first step of the process according to the invention, a combination of the carbohydrate matrix ingredient and a viscosity modifying ingredient is provided and mixed in the extruder.

The carbohydrate matrix comprises a starch with an amylopectin content of at least 80%. The starch used with an amylopectin content of at least 80% can be used directly and without further pretreatment.

The term “starch” includes both native starch(es) and modified starch(es), i.e., starch products obtained by physical, chemical or enzymatic processes, from different starch sources.

In a preferred embodiment of the process according to the invention, the starch having an amylopectin content of at least 80% is selected from the group consisting of rice starch, potato starch and combinations of the two mentioned starches.

Most preferred is starch with an amylopectin content of at least 90%.

The most suitable is amylopectin potato starch for the formation of the carbohydrate matrix. Amylopectin potato starch is based on natural breeding and has an amylopectin content of at least 95% and contains virtually no amylose. Such an amylopectin potato starch is commercially available, for example, under the name “Eliane™ Gel 100”. Amylopectin potato starch is characterized by high purity, particular process stability and very high viscosity.

In a preferred embodiment, the starch has a viscosity of 10 mPa·s to 50.000 mPa·s, preferably a viscosity of 20 mPa·s to 30.000 mPa·s, measured as a 10% solution in water at 90° C. using a rheometer (Anton Paar Rheometer MCR302, cone-plate system, cone type CP-50-1 at a constant shear rate of 5 sec⁻¹).

Most preferred for the preparation of the color particles according to the present invention are starches from potatoes and rice having a viscosity of 10 mPa·s to 50.000 mPa·s, preferably a viscosity of 20 mPa·s to 30.000 mPa·s, measured as a 10% solution in water at 90° C. using a rheometer (Anton Paar Rheometer MCR302, cone-plate system, cone type CP-50-1 at a constant shear rate of 5 sec¹).

The viscosity modifying component lowers the softening point of the matrix: The higher its proportion in the mixture, the easier it is to extrude the carbohydrate matrix. Furthermore, this ingredient facilitates granulation at the perforated die.

In the second step of the process according to the invention, the further components of the color particles according to the invention, such as emulsifier, water and optionally colorant, flavoring and/or odiferous substance or aroma and/or scent, are continuously fed to the extruder in the dosages described above.

Alternatively, the two steps (first step and second step) can also be combined. For this purpose, all components are mixed beforehand and then continuously fed to the extruder.

The at least one emulsifier causes the carbohydrate matrix and the other components of the color particles to emulsify, suspend or form a gel-like structure with each other. The emulsifier also promotes transport of the materials during extrusion, reduces adhesion to the walls of the extruder, and improves transport of the mixture through the dies of the extruder.

The addition of water causes a lowering of the glass transition temperature, and the plasticizing effect of water favors the thermoplastic extrusion of the carbohydrate matrix. During the extrusion of starch, the shear forces destroy the starch granules, and water can penetrate the starch molecules more quickly and trigger gelatinization. Extrusion with water produces a thermoplastically deformable mixture in the extruder that exhibits pronounced viscoelasticity.

In an alternative variant of the process according to the present invention, all components of the color particles, i.e. carbohydrate matrix comprising or consisting of a starch with an amylopectin content of at least 80%, at least one viscosity modifying component, an emulsifier, water and optionally at least one colorant and/or at least one flavoring or odiferous substance and/or at least one aroma or scent are provided and mixed in one process step to obtain a highly viscous mixture.

With regard to the constituents of the color particles, their preferred or alternative embodiments, their mixing and quantity ratios and their advantageous effects, reference is made to the above detailed description in connection with the color particles according to the invention, which is equally valid for the process according to the invention in accordance with the second aspect of the invention, so that it is unnecessary to repeat it.

After completion of the mixing and dispersing or suspending of the components of the color particles according to the invention in the process according to the invention, a highly viscous mixture is present in the form of a suspension or gel, which is extruded in a further step, thereby obtaining an extrudate.

The extrudate is continuously pressed through the die(s) of the extruder. As a result, strands are formed downstream of the die opening. The diameter of the strands and thus the diameter of the color particles finally obtained is controlled via the die diameter. A minimum pressure of 1 bar must be built up in front of the nozzle to ensure uniform discharge without pulsing. The ideal pressure range is between 1 and 60 bar, preferably from 4 to 50 bar and particularly preferably from 5 to 45 bar.

Particularly good producibility in the extruder is achieved when starch with an amylopectin content of more than 90%, in particular pure amylopectin potato starch, is used in the process according to the invention to form the carbohydrate matrix. The amylopectin potato starch with more than 95% amylopectin content contains practically no amylose. Such an amylopectin potato starch is commercially available, for example, under the name “Eliane™ Gel 100”.

Since the viscosity of the suspension or gel has an influence on the stability of the color particles during application, in the process according to the present invention, in order to achieve the best possible stability of the color particles, a starch is used which has a viscosity of 10 to 50.000 mPa·s, measured as a 10% solution in water at 90° C.

Preferably, a twin-screw or twin-shaft extruder is used for mixing the components of the color particle according to the invention, which can be equipped with several temperature zones so that the temperature during mixing and extrusion can be specifically controlled. The viscosity of the mixture is also controlled by heating the extruder housing and the frictional heat of the screw rotations.

Mixing of the components of the color particle according to the invention and/or extrusion of the suspension or gel is carried out at an elevated temperature. The increased temperature affects the viscosity of the suspension or gel, which is thereby lowered. This makes it easier to force the suspension or gel through the die plate of the extruder. Preferably, mixing of the components of the color particle according to the invention and/or extrusion of the suspension or gel is carried out at a temperature in the range from 70 to 150° C., preferably from 75 to 130° C. and particularly preferably from 80 to 120° C.

The torque occurring in the extruder during extrusion (measured in % of the max. torque or the total torque of the extruder) represents a measure of the producibility. If the torque is in the range of 30-60%, the color particles can be produced well i.e., uniform particles are produced during granulation at the head of the extruder and the extrusion runs for several hours without technical malfunction. A Leistritz ZSE 18MAXX twin-screw extruder was used for the experiments in the present invention. According to the manufacturer, this has a total screw torque of 71 Nm.

A suitable screw configuration controls the degree of filling and the residence time in the extruder. The screw speed can be used to control the degree of filling, the mixing efficiency, the frictional heat generated and the material pressure.

To form the strands after opening, the extruded compound is cooled.

The strands can be comminuted by means of a cold or hot die cutting process. Advantageously, the strands are comminuted while still in the solidification phase by head pelletizing or a hot die cutting process. For this purpose, a head pelletizer, which may be gas-tight, with rotating cutting knives is preferably used, which takes place directly at the outlet of the extruder. A (stepless) speed control of the head pelletizer allows the particle length to be adjusted as a function of the solids throughput.

Depending on the moisture content, the color particles thus obtained are still dried if necessary.

The bulk density of the color particles according to the invention is 500-1,000 g/l; particularly preferably, the bulk density is 600-900 g/l.

In a further, third aspect, the present invention relates to color particles comprising or consisting of a carbohydrate matrix comprising or consisting of a starch having an amylopectin content of at least 80% and at least one viscosity modifying ingredient, at least one emulsifier, water, and optionally at least one colorant and/or at least one flavoring or odiferous substance or at least one aroma or scent obtainable by the process according to the invention.

Preferably, the process is carried out with a starch containing more than 90% amylopectin, most preferably with a starch containing more than 95% amylopectin that has practically no amylose.

A further aspect of the invention relates to the use of the color particles according to the invention for the production of foods, beverages, cosmetics, in particular oral hygiene products, pharmaceuticals, consumer goods, in particular detergents and cleaning agents in liquid, gel or powder form, pet food or pet care products.

Due to their advantageous properties, the color particles according to the invention are excellently suited for giving the above-mentioned products a visually improved appearance or texture. A significant advantage of the color particles according to the invention is that, due to their stability, the optical appearance or texture can be maintained over the shelf life of the products.

Most preferably, the color particles according to the invention are used in oral care products such as chewing gums, toothpaste, mouth gels, chewable tablets and chewy sweets, and cosmetic products such as shampoo, shower gel, exfoliating products, creams, lotions, foodstuffs, in particular dairy products such as yogurt, beverages, and detergents and cleaning products such as dishwashing products, liquid soaps, liquid washing emulsions, soaps or toilet cleaners.

With regard to preferred constituents of the color particles, their preferred or alternative embodiments, their mixing and quantity ratios and their advantageous effects, reference is made to the above detailed description in connection with the color particles according to the invention, which is equally valid for the process according to the invention in accordance with the second aspect of the invention, so that it is unnecessary to repeat it.

Particularly preferred for use according to the invention are color particles comprising starch with an amylopectin content of more than 90%, in particular potato starch or rice starch.

The best results are obtained with color particles comprising pure amylopectin potato starch to form the carbohydrate matrix. This starch with over 95% amylopectin content contains virtually no amylose.

Ultimately, the present invention relates to foodstuffs, beverages, cosmetics, in particular oral hygiene products, pharmaceuticals, consumer articles, in particular detergents and cleaning agents in liquid, gel or powder form, pet food or pet care products, which contain the color particles according to the invention.

In a preferred embodiment, the food products are selected from the group consisting of instant beverage powders, tea, soup or sauce powders, baked goods, chewy sweets, confectionery and dairy products, preferably yogurt. The color particles are most preferably used to improve the appearance and consistency of the following products: pre-shave products, acidic, alkaline and neutral detergents, e.g. floor cleaners, window glass cleaners, dishwashing detergents, bathroom and sanitary cleaners, scouring agents, solid and liquid WC cleaners, liquid detergents, powder detergents, fabric softeners, laundry soaps, disinfectants, air fresheners in liquid or gel form, personal care products such as solid and liquid soaps, shower gels, shampoos, shaving soaps, shaving foams, bath oils, cosmetic emulsions of the oil-in-water, water-in-oil and water-in-oil-in-water type, e.g., skin creams and lotions, facial creams and lotions, sunscreen creams and lotions, after-sun creams and lotions, hand creams and lotions, foot creams and lotions, depilatory creams and lotions, aftershave creams and lotions, tanning creams and lotions, hair care products such as hair gels, hair lotions, hair conditioners, hair creams and lotions, deodorants and antiperspirants, such as underarm sprays, roll-ons, deodorant sticks, deodorant creams, and decorative cosmetics and oral hygiene products.

Most preferred are the cosmetics selected from the group that consists of oral care products such as chewing gum, toothpaste, mouth gels, chewing tablets and chewing candies, shampoo, shower gel, exfoliating products, creams and lotions.

According to a preferred further development of the invention, the color particles according to the present invention are present in these foodstuffs, cosmetics, in particular oral hygiene products, pharmaceuticals, consumer articles, in particular detergents and cleaning agents in liquid, gel or powder form, animal foodstuffs or animal care products in amounts of 0.001 to 10% by weight, even more preferably 0.05 to 4% by weight and most preferably 0.1 to 2% by weight, based on the total weight of the preparation.

The present invention is described in more detail below by means of examples which, however, do not limit the scope of protection of the objects according to the invention. Unless otherwise indicated, the proportions given below refer to weight.

### EXAMPLES

### Example 1

Preparation of the Color Particles According to the Invention

A suitable raw material compound, as described in the following examples, was metered into a Leistritz ZSE 18-MAXX twin-screw extruder with eight-barrel blocks and several separately temperature-controlled zones and processed with the following operating conditions. The temperature profile in the barrels was controlled as follows: housing 1: unheated; housings 2 to 8: 90° C.; die plate (1 mm hole diameter): 90° C. At a screw speed of 100 rpm, the working pressure was 9 bar; the throughput was 2 kg/h.

For the production of color particles with a nominal particle size of approx. 1 mm, 1 to 2 knives made of sheet steel and a pelletizing plate with 18 holes with a nominal diameter of 1.0 mm were used. The speed control of the head pelletizer for setting the pellet length was stepless depending on the solids throughput. The granules obtained have a bulk density of approx. 600 to 900 g/liter. Subsequently, dust and oversize particles were removed by a double-deck sieve with sieve sizes of 0.8 mm and 1.25 mm. The sieve losses amounted to less than 5% of the yield.

The color particles thus obtained were added to the toothpaste bases prepared as described below at a dosage of 1% by weight or to a commercial yogurt, 1.5% fat content, at a dosage of 1% by weight.

### Example 2

Preparation of a Toothpaste Base (Silica Base)

| Block A | Weight % |
|---|---|
| Water, demin. | 21.81 |
| Sorbitol 70% | 45.00 |
| Solbrol M Na-salt | 0.15 |
| Trisodium phosphate | 0.10 |
| Saccharin 450-fold | 0.20 |
| Sodium monofluorophosphate | 1.14 |
| PEG 1500 | 5.00 |

| Block B | Weight % |
|---|---|
| Sident 9 (abrasive silica) | 10.00 |
| Sident 22 S (thickening silica) | 8.00 |
| Sodium carboxymethyl cellulose | 1.10 |
| Titanium (IV) oxide | 0.50 |

| Block C | Weight % |
|---|---|
| Water, demin. | 4.50 |
| Sodium lauryl sulfate (SLS) | 1.50 |

The ingredients of block A were mixed and added to a blender. The ingredients of block B were mixed, added to the ingredients of block A in the mixer, mixed under vacuum at 25 to 30° C. for 30 min, then brought to normal pressure and the mixer was stopped. The ingredients of block C were mixed and added to the mixture in the mixer, mixed under vacuum at 25 to 30° C. for 20 to 30 min, then brought to normal pressure and the mixer was stopped.

### Example 3

| Block A | Weight % |
|---|---|
| Water, demin. | 21.00 |
| Saccharin 450-fold | 0.20 |
| Solbrol M Na-Salt | 0.20 |
| Sodium monofluorophosphate | 0.80 |
| Sorbitol 70% | 29.00 |

| Block B | Weight % |
|---|---|
| Calcium carbonate | 35.00 |
| Sident 22 S (thickening silica) | 3.50 |
| Sodium carboxymethyl cellulose | 1.30 |
| Titanium (IV) oxide | 0.50 |

| Block C | Weight % |
|---|---|
| Water, demin | 5.50 |
| Sodium Lauryl Sulfate | 2.00 |

The ingredients of block A were mixed and added to a blender. The ingredients of block B were mixed, added to the ingredients of block A in the mixer, mixed under vacuum at 30-35° C. for 45 min, then brought to normal pressure and the mixer was stopped. The ingredients of block C were mixed and added to the mixture in the mixer, mixed under vacuum at 30-35° C. for 20 min, then brought to normal pressure and the mixer was stopped.

### Example 4

Making a Toothpaste Base (Gel Base)

| Block A | Weight % |
|---|---|
| Sorbitol 70% | 63.00 |
| Water, demin. | 6.81 |
| Saccharin | 0.20 |
| Sodium monofluorophosphate | 1.14 |
| Solbrol | 0.15 |
| Trisodium phosphate | 0.10 |
| PEG 1500 (PEG 32) | 5.00 |

| Block B | Weight % |
|---|---|
| Sident 9 (abrasive Silica) | 8.00 |
| Sident 22 S (thickening silica) | 8.00 |
| Sodium carboxymethyl cellulose | 0.60 |

| Block C | Weight % |
|---|---|
| Water, demin. | 4.50 |
| Sodium Lauryl Sulfate | 1.50 |

The ingredients of block A were mixed and put into a mixer. The ingredients of block B were mixed, added to the ingredients of block A in the mixer, mixed under vacuum at 40-45° C. for 15 min, then brought to normal pressure and the mixer was stopped. The ingredients of block C were mixed and added to the mixture in the mixer, mixed under vacuum at 40-45° C. for 15 min, then brought to normal pressure and the mixer was stopped.

TABLE 2
Application test in toothpaste bases with a dosage of 1 wt. %.

| | Application test in toothpaste | | |
|---|---|---|---|
| | (3 weeks at 45° C.) | | |
| Starch component of | Gel base | Silica base | Carbonate base |
| the color particle | (according to | (according to | (according to |
| formulation | example 4) | example 2) | example 3) |
| Potato starch | O.K. | O.K. | O.K. |
| (Eliane ™ Gel 100) | | | |
| Rice starch native | O.K. | O.K. | O.K. |
| Potato starch | O.K. | O.K. | O.K. |
| (Aero-Myl) | | | |
| Wheat starch | not O.K. | not O.K. | not O.K. |
| | very soft | | |
| Potato starch | O.K. | O.K. | O.K. |
| (Paselli ™) | | | |
| Capsul ® Tapioca | not O.K.; | not O.K.; | not O.K.; |
| | Particles | Particles | Particles |
| | dissolve when | dissolve when | dissolve when |
| | squeezed out | squeezed out | squeezed out |

Note:
O.K. = okay; corresponds to stably detectable color particles in the toothpaste base.

TABLE 3
Application test in commercial yogurt,
1.5% fat, with a dosage of 1 wt. %.

| | |
|---|---|
| | Stability in yogurt |
| | (1% granules in white yogurt; |
| Starch component of | 3 days storage in the refrigerator) |

| the color particle | optical | mechanical |
|---|---|---|
| formulation | (color bled out?) | (easy to rub in?) |
| Rice starch native | Particles detectable; | Perceptible as |
| | not bled out | particles |
| Potato starch | Particles detectable; | Perceptible as |
| (Aero-Myl) | not bled out | particles |
| Wheat starch | Particles bled out; | |
| | no particles detectable | |
| | after stirring | |
| Potato starch | Particles detectable; | After rubbing no |
| (Paselli ™) | not bled out | longer perceptible |
| | | as particles |
| Capsul ® Tapioca | Particles bled out; | |
| | No particles detectable | |
| | after stirring | |
| Potato starch | Particles detectable; | Perceptible as |
| (Eliane ™ Gel 100) | not bled out | particles |

As a comparison of the color particles according to the invention produced from rice or potato starch with color particles produced from corn, wheat or tapioca starch shows, the color particles according to the invention are characterized by good storage stability over a longer period of time and good producibility in the extruder. Even in an application with a (high) water content, the color particles according to the invention do not bleed out or dissolve.

### Example 5

Production of Color Particles with Potato Starch

| Designation | Quantity (g) |
|---|---|
| Potato starch (Eliane ™ Gel 100) | 721 |
| Sorbitol | 200 |
| Water | 30 |
| Emulsifier monoglycerides | 17 |
| Cosmetic colorant blue CI74160 | 2 |
| Mint aroma Optacool ® OP | 30 |
| Sum: | 1,000 |

The raw materials were mixed and metered into the extruder. The color particles were produced by means of a laboratory extruder (twin screw extruder, Leistritz ZSE 18 MAXX). The temperature profile in the housings is controlled as follows: housing 1: unheated; housing 2 to 9: 90° C.; die plate (hole diameter 1 mm): 90° C. At a screw speed of 100 rpm, the working pressure was 9 bar; the throughput was 2 kg/h.

(A) Application in Water

The color particles were added to water (1 g in 20 ml water) and evaluated after one week.

As such, the color particles were well preserved; they were not bled out and the surrounding water showed no visible turbidity. The color particles were readily spreadable and therefore suitable for the application described above.

(B) Application in Toothpaste

The color particles were applied in different toothpaste bases at a dosage of 1 wt. %, stored at 45° C. and evaluated after three weeks. Three toothpaste bases (silica base, carbonate base, gel base) prepared as described in examples 2 to 4 served as toothpaste bases.

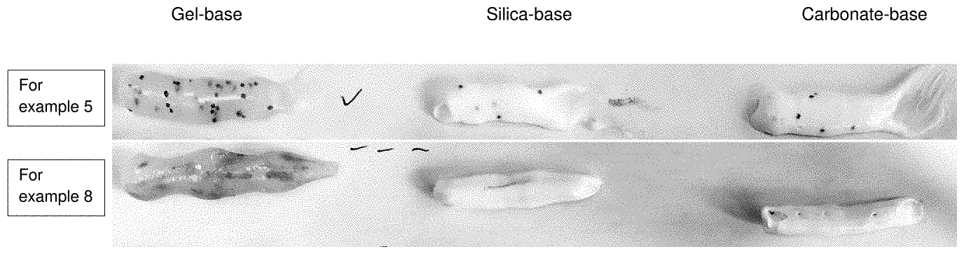

As shown by the first row of toothpaste samples illustrated in FIG. 1 in the order gel base, silica base and carbonate base, the color particles as such were well preserved in all three toothpaste samples; the surrounding toothpaste base did not exhibit any visible bleeding, as can be seen in FIG. 1. The color particles were readily spreadable and therefore suitable for the application described above.

### Example 6

Production of Color Particles with Potato Starch

| Designation | Quantity (g) |
|---|---|
| Potato starch (Eliane ™ Gel 100) | 768 |
| Dextrin from corn (Nutriose ®) | 100 |
| Water | 100 |
| Emulsifier monoglycerides | 17 |
| Food coloring | 5 |
| Titanium dioxide E171 | |
| Mintaroma Optamint ® | 10 |
| Sum: | 1,000 |

The raw materials were mixed and metered into the extruder. The color particles were produced by means of a laboratory extruder (twin screw extruder, Leistritz ZSE 18 MAXX). The temperature profile in the housings is controlled as follows: housing 1: unheated; housing 2 to 9: 90° C.; die plate (hole diameter 1 mm): 90° C. At a screw speed of 100 rpm, the working pressure was 9 bar; the throughput was 2 kg/h.

(A) Application in Water

The color particles were added to water (1 g in 20 ml water) and evaluated after one week.

As such, the color particles were well preserved; they were not bled out and the surrounding water showed no visible turbidity. The color particles were readily spreadable and therefore suitable for the application described above.

(B) Application in Toothpaste

The color particles were applied in different toothpaste bases at a dosage of 1 wt %, stored at 45° C. and evaluated after three weeks. The toothpaste bases used were the three toothpaste bases described in Examples 2 to 4 (silica base, carbonate base, gel base).

The color particles were well preserved as such; the surrounding toothpaste base showed no visible bleeding. The color particles were readily spreadable and therefore suitable for the application described above.

### Example 7

Production of Color Particles with Corn Starch

| Designation | Quantity (g) |
|---|---|
| Cornstarch | 774 |
| Dextrin from corn (Nutriose ®) | 100 |
| Water | 100 |
| Emulsifier Monoglycerides | 11 |
| Food coloring | 5 |
| Titanium dioxide E171 | |
| Mint flavor Optacool ® MG-E | 10 |
| Sum: | 1,000 |

The color particles were prepared as described in Example 6.

(A) Application in Water

The color particles were added to water (1 g in 20 ml water) and evaluated after one week. The color particles had become a cloudy mass and were no longer perceptible as particles.

### Example 8

Production of Color Particles with Capsul® Tapioca

| Designation | Quantity (g) |
|---|---|
| Capsul ® Tapioca | 751 |
| Dextrin from corn (Nutriose ®) | 100 |
| Water | 100 |
| Emulsifier Monoglycerides | 17 |
| Cosmetic Colorant Blue CI74160 | 2 |
| Mint aroma Optacool ® OP | 30 |
| Sum: | 1,000 |

The color particles were prepared as described in Example 3.

(A) Application in Water

The color particles were added to water (1 g in 20 ml water) and evaluated after one week. The color particles had become a cloudy mass and were no longer perceptible as particles.

(B) Application in Toothpaste

The color particles were applied in different toothpaste bases at a dosage of 1 wt %, stored at 45° C. and evaluated after three weeks. Three toothpaste bases (silica base, carbonate base, gel base) were used as toothpaste bases, which were prepared as described in Examples 2 to 4.

As shown by the second row of toothpaste samples shown in FIG. 1 in the order gel base, silica base and carbonate base, the color particles were no longer discernible. The surrounding toothpaste base showed clear bleeding. The color particles are therefore not suitable for the application described above.

### Example 9

Analysis of the Viscosity of Different Starches

The starch powders were each prepared as a 10% suspension in water at room temperature.

An Anton Paar Rheometer MCR302, cone-plate system, cone type CP-50-1 at a constant shear rate of 5 sec⁻¹ was used to determine viscosity. Investigations were carried out in a temperature range of 25-90° C.: (i) heating: temperature program 25-90° C. with a heating rate of 13° C. per min, and (ii) cooling: temperature program 90-25° C. with a cooling rate of 13° C. per min. The results of the measurements are given in Table 4 below:

TABLE 4

| | | Viscosity | Viscosity |
|---|---|---|---|
| | Temperature | (heating) | (cooling) |
| Raw material | (° C.) | (mPa · s) | (mPa · s) |
| Potato starch | 90 | 19 | 20 |
| (Eliane ™ Gel | 80 | 53 | 15 |
| 100) | 70 | 193 | 17 |
| | 60 | | 21 |
| | 50 | | 30 |
| | 40 | | 46 |
| Cornstarch | 90 | 181,200 | 170,700 |
| | 80 | 274,300 | 179,600 |
| | 70 | 91,890 | 205,900 |
| | 60 | | 248,200 |
| | 50 | | 300,200 |
| | 40 | | 377,000 |
| Wax corn | 90 | 221 | 352 |
| Capsul ® | 80 | 2 | 2,425 |
| | 70 | 2 | 1,453 |
| | 60 | | 626 |
| | 50 | | 416 |
| | 40 | | 274 |
| Potato starch | 90 | 491 | 1,046 |
| (Paselli ™) | 80 | 3 | 2,107 |
| | 70 | 2 | 1,601 |
| | 60 | | 389 |
| | 50 | | 391 |
| | 40 | | 111 |
| Potato starch | 90 | 110 | 185 |
| (Aero-Myl) | 80 | 48 | 721 |
| | 70 | 45 | 783 |
| | 60 | | 983 |
| | 50 | | 875 |
| | 40 | | 908 |
| Rice starch | 90 | 31,340 | 28,490 |
| | 80 | 46,400 | 26,620 |
| | 70 | 186 | 28,710 |
| | 60 | | 30,420 |
| | 50 | | 34,280 |
| | 40 | | 37,510 |
| Wheat starch | 90 | 87,740 | 88,270 |
| | 80 | 100,800 | 96,850 |
| | 70 | 124,200 | 108,800 |
| | 60 | | 129,700 |
| | 50 | | 158,000 |
| | 40 | | 207,100 |
| Capsul ® | 90 | 4 | 4 |
| Tapioca | 80 | 3 | 3 |
| | 70 | 3 | 2 |
| | 60 | | 2 |
| | 50 | | 2 |
| | 40 | | 2 |

The viscosity of the starch has an influence on the stability of the color particles during application. Preferably, the viscosity of the starch used is 10 mPa·s to 50.000 mPa·s, measured as a 10% solution in water at 90° C.

1. Color particles comprising or consisting of an extrudate from an extrusion process, wherein the extrudate comprises:
a carbohydrate matrix comprising a starch having an amylopectin content of at least 80% and at least one viscosity modifying component;
at least one emulsifier; and
water.

2. Color particles, according to claim 1, wherein the starch has an amylopectin content of at least 90%.

3. Color particles, according to claim 1, wherein the starch is selected from potato starch or rice starch or a combination thereof; and/or wherein the starch has a viscosity of from 10 mPa·s to 50,000 mPa·s, measured as a 10% solution in water at 90° C. using a viscometer.

4. Color particles, according to claim 1, wherein the at least one viscosity-modifying ingredient is a water-soluble compound selected from one or more in the group of mono, di and trisaccharides, sugar alcohols, dextrins or maltodextrins, and mixtures of the aforementioned compounds.

5. Color particles, according to claim 1, wherein the sugar alcohol is selected from one or more in the group of sorbitol, mannitol, isomalt, lactitol, xylitol, threitol, erythritol, arabinol, arabitol, adontol, alditol, ducitol, iditol and mixtures of the aforementioned sugar alcohols.

6. Color particles, according to claim 1, wherein the ratio of starch having an amylopectin content of at least 80% to the viscosity modifying ingredient ranges from 99:1 to 50:50.

7. Color particles, according to claim 1, wherein the at least one emulsifier is selected from one or more in the group of: monoglycerides, diglycerides, deca-glycerol dipalmitate, hexa-glycerol distearate, polyglycerol esters, sulfoacetates, lecithin, polysorbates and mixtures of the aforementioned emulsifiers.

8. Color particles, according to claim 1, wherein the at least one colorant is selected from one or more in the group of oil-soluble colorants and pigment colorants: E141, E153, E160, E160a, E160b, E160c, E171, E172, CI11680, CI12085, CI12490, CI13015, CI15850, CI16185, CI18965, CI19140, CI42045, CI42051, CI42090, CI45350, CI45410, CI47005, CI59040, CI60725, CI61565 CI61570, CI74160, CI74260, CI77007, CI77019, CI77266, CI73360, CI77492, CI77499, CI77891 and mixtures of the abovementioned colorants or pigment colorants.

9. Color particles, according to claim 1, comprising:
50 to 98% by weight of a carbohydrate matrix of a starch having an amylopectin content of at least 80% and at least one viscosity modifying component;
0.1 to 5% by weight of at least one emulsifier; and
1 to 30% by weight of water;
based on the total weight of the color particle.

10. Color particle, according to claim 1, wherein the water content is 1 to 30% by weight, based on the total weight of the color particle.

11. Color particles, according to claim 1, wherein the particles have a particle diameter in a range from 0.2 to 5 mm, and/or a length in a range from 0.1 to 10 mm.

12. A method of making a color particle comprising:
providing a combination of a carbohydrate matrix comprising a starch having an amylopectin content of at least 80% and at least one viscosity modifying component;
preparing a mixture of the combination obtained from step (i) and at least one emulsifier, water to obtain a suspension or gel;
extruding the suspension or gel to obtain an extrudate; and
comminuting the extrudate to obtain a color particle.

13. The process for producing the color particle according to claim 12, wherein the extrusion of the emulsion is carried out at a temperature in the range of 70 to 150° C. and/or at a pressure of 1 to 60 bar and/or the torque occurring in the extruder during extrusion is in the range of 30-60%, based on the max. torque of the extruder.

14. Color particles obtainable by the method of claim 12.

15. (canceled)

16. A foodstuff, beverage, cosmetic, oral hygiene, pharmaceutical, consumer article, detergent, cleaning agent pet food, or pet care product comprising color particles according to claim 1.

17. The product of claim 16, wherein the food product is selected from the group of: instant beverage powders, tea, soup or sauce powders, bakery products, chewy candies, confectionery, dairy products; the cosmetic product is selected from the group of oral care products, chewing gums, toothpaste, oral gels, chewable tablets, shampoo, shower gel, exfoliating products, creams, and lotions; and the cleaning product is selected from the group of dishwashing detergents, liquid soaps, liquid washing emulsions, and soaps.

18. Colored particles, according to claim 1, further comprising at least one colorant.

19. Colored particles, according to claim 1, further comprising at least one flavoring substance.

20. Colored particles, according to claim 1, further comprising at least one or odiferous, aroma or scent substance.
